(19) United States
(12) Patent Application Publication
(10) Pub. No.: US 2013/0302424 A1
(43) Pub. Date: Nov. 14, 2013

(54) COMPOSITION FOR PREVENTING AND TREATING MASTITIS

(71) Applicant: Shintaro Inoue, Tokyo (JP)

(72) Inventor: Shintaro Inoue, Tokyo (JP)

(73) Assignee: FLORAL MINERAL CORPORATION RESEARCH LABORATORY, Shinjuku-ku, Tokyo, (JP)

(21) Appl. No.: 13/978,649

(22) PCT Filed: Jun. 21, 2012
(86) PCT No.: PCT/JP2012/065801

§ 371 Date: Jul. 8, 2013

(30) Foreign Application Priority Data
Jul. 12, 2011 (JP) 2011-153695

(51) Int. Cl. A61K 33/08; A61K 33/00

(52) U.S. Cl. CPC A61K 33/08; A61K 33/00
USPC 424/489; 424/691

(57) ABSTRACT

The purpose of the present invention is to reduce the amounts of nitrate nitrogen and ammonia, which can be absorbed through the digestive tract and are factors for causing mastitis in livestock animals, thereby reducing the incidence rate of mastitis. A composition for preventing and treating mastitis for livestock animals comprises a rock powder containing silicic acid and aluminum oxide as active ingredients. When the composition is mixed with feed and the feed is given to livestock animals, nitrogen compounds including ammonia nitrogen and nitrate nitrogen generated in the body of the livestock animals can be chemically adsorbed by aluminum oxide and silicic acid, whereby the over-absorption of the nitrogen compounds through the digestive tract can be suppressed. At the same time, minor elements contained in the rock powder act to fully elicit the activity of a nutritional component capable of suppressing mastitis, e.g., vitamin A, vitamin E, copper, zinc and manganese obtained from the feed. In this manner, the onset of mastitis can be suppressed in a nutritional manner, and a therapeutic effect can be achieved on mastitis that has already been developed.

### TECHNICAL FIELD

The present invention relates to a composition in dairy farming art for preventing and treating mastitis in livestock animals to be milked such as dairy cattle.

### BACKGROUND ART

In general, speaking of mastitis in livestock animals, it is a disease which livestock animals such as milked cattle and milked goats suffer from, and for dairy farmers, it is a disease which reduces the amount of lactation or which becomes a factor in remarkably lowering their production efficiency and management efficiency since the milk cannot be sold. Moreover, there are elements for causing mastitis such as sanitary conditions (pathogen) of the environment where the cattle are kept, feed, stress, and genetic elements.

In recent years, the amount of lactation per animal is large so that the animals bear a load on their udder and easily get mastitis. Moreover, high-calorie and high-protein feed is given to the animals in order to increase the amount of their lactation, thereby generating a large amount of nitrate nitrogen and ammonia in their rumen. Such a large amount of nitrate nitrogen and ammonia absorbed through their digestive tract also becomes a factor in increasing mastitis.

Now, the dairy farmers give the livestock animals antibacterial substances (antibiotics) as practical coping strategy when the cattle or milked livestock animals get mastitis. In this case, the milk cannot be sold if the antibiotics remain in the milk, and unless a certain period of time has passed after the antibiotics were administered, the meat of the livestock animals cannot be sold even if they are slaughtered. This also remarkably lowers their productivity and management efficiency. Therefore, a plurality of inventions for preventing or treating mastitis without the use of antibiotics are publicly known as prior art.

The first prior art according to the publicly known inventions is, for example, a composition for preventing and treating mastitis in livestock animals which includes viable baker's yeast cells or inclusion thereof as active ingredients, and the above described inclusion is at least one selected from baker's yeast, moromi, concentrates, pasted materials, dry matters, and dilutions (see Patent Literature 1).

In this composition for preventing and treating mastitis in livestock animals, mastitis can be prevented and/or treated by orally administering (feeding 0.5 g to 200 g of) active viable baker's yeast cells to the livestock animals. As a result, various and great losses such as decrease in milk production quantities, decline in quality of the milk, cost for treating the cattle, a loss by selection can be prevented, and a very tremendous economic effect can be brought about to the dairy farmers.

Moreover, the second prior art according to the publicly known inventions is, for example, is a preventive agent for mastitis for livestock animals which contains a nitrogen-containing compound (at least one selected from betaine, choline, methionine, S-adenosylmethionine, vitamin B₁₂, folic acid, and carnitine) that acts as a methyl group donor in vivo and a fat-soluble vitamin (at least one selected from vitamin A, β-carotene, and vitamin E) as active ingredients (see Patent Literature 2).

This preventive agent for mastitis for livestock animals can efficiently prevent/treat mastitis by feeding both ingredients of amino acids with a methyl group and fat-soluble vitamins to the livestock animals simultaneously or separately. For example, in the case of dairy cattle, a good effect was confirmed by administering about 50 g of betaine and about 1 g of vitamin E (less than 50%) (as vitamin E, about 500 mg, i.e. about 500 IU) per day to the dairy cattle, thereby preventing various and great economic losses due to mastitis, such as decrease in milk production quantities, decline in quality of the milk, milk disposal, a loss due to cow selection, the purchase cost of substitute cows, drug cost, medical expense paid to a veterinarian, and feedstuff cost while milk cannot be shipped, and bringing about a very tremendous economic effect to the dairy farmers.

Furthermore, the third prior art according to the publicly known inventions is a preventive agent for mastitis in livestock animals which contains one or more selected from the group consisting of carboxylic acids (one or more selected from citric acid, malic acid, fumaric acid, tartaric acid, lactic acid, gluconic acid, succinic acid, formic acid, acetic acid, propionic acid and butyric acid) and their salts (one or more selected from sodium salt, potassium salt, calcium salt, magnesium salt, copper salt, zinc salt, ammonium salt, iron salt, cobalt salt and cerium salt of carboxylic acids) as active ingredients (see Patent Literature 3).

Since this preventive agent for mastitis is a preventive agent for mastitis in livestock animals which contains carboxylic acids and their salts as active ingredients, it is an inexpensive and highly safe preventive agent for mastitis in livestock animals. Moreover, since it can be mixed with mixed feed and the like and administered by oral administration, it is a simple and extremely practical preventive agent for mastitis in livestock animals.

### CITATION LIST

### Patent Literature

### Patent Literature 1

Japanese Patent Laid-Open No. 2001-224317

### Patent Literature 2

Japanese Patent Laid-Open No. 2003-183180

### Patent Literature 3

Japanese Patent Laid-Open No. 2005-41798

### SUMMARY OF INVENTION

### Technical Problem

Although the above described first prior art includes viable baker's yeast cells or inclusion thereof as active ingredients, it does not reveal what a factor for causing mastitis is. Moreover, it does not disclose and it is unclear how viable baker's yeast cells act on that factor for causing mastitis to prevent or treat mastitis.

Moreover, similar to the above described first prior art, also the above described second prior art does not reveal what a factor for causing mastitis is. Also, it does not disclose and it is unclear how the betaine and vitamin E act on that factor for causing mastitis to prevent or treat mastitis.

Furthermore, although the above described third prior art is a preventive agent for mastitis for livestock animals which contains carboxylic acids and their salts as active ingredients, it does not explain at all how the carboxylic acids and their salts act on mastitis or a factor for causing mastitis to prevent or treat mastitis. Therefore, its effect is substantially ununderstandable and unclear.

Thus, it is a problem to be solved that the incidence rate of mastitis is decreased by reducing the amount of nitrate nitrogen and ammonia absorbed through their digestive tract which are factors for causing mastitis.

### Solution to Problem

The present invention is intended to provide a composition for preventing and treating mastitis for livestock animals, wherein the composition comprises a rock powder containing silicic acid and aluminum oxide as active ingredients as a specific means for solving the above mentioned problem.

The present invention includes the following additional conditions that: the silicic acid content is 60% or more and the aluminum oxide content is in the range of 6 to 12%; the ratio of a value obtained by dividing the silicic acid content by the molecular weight of the silicic acid to a value obtained by dividing the aluminum oxide content by the molecular weight of the aluminum oxide is more than 10; the above described rock powder has a three-layered structure of a silicic acid layer-an alumina layer-a silicic acid layer; the above described rock is shale which mainly includes sediment from organisms; and the above described powder of the active ingredients is in the form of granules or pellets.

### Advantageous Effects of Invention

The composition for preventing and treating mastitis for livestock animals according to the present invention comprises a rock powder containing silicic acid and aluminum oxide as active ingredients. When the composition is mixed with feed and the feed is given to livestock animals, nitrogen compounds including ammonia nitrogen and nitrate nitrogen generated in the body of the livestock animals can be chemically adsorbed by the aluminum oxide and the silicic acid, whereby the over-absorption of the nitrogen compounds through the digestive tract can be suppressed. At the same time, minor elements contained in the silicic acid act to fully elicit the activity of a nutritional component capable of suppressing mastitis, e.g. vitamin A, vitamin E, copper, zinc and manganese obtained from the feed. In this manner, the present invention exhibits an excellent effect that the onset of mastitis can be suppressed in a nutritional manner, and a therapeutic effect can be achieved on mastitis that has already been developed.

### DESCRIPTION OF EMBODIMENTS

The present invention will be explained in detail on the basis of the following embodiments. The composition for preventing and treating mastitis for livestock animals according to the present invention is a rock powder in which silicic acid and aluminum oxide are contained with the required ratio (silica-alumina ratio) as active ingredients. An effectively preferred condition is that the silica-alumina ratio (ratio of silicic acid and aluminum oxide) in this case is more than 10 and the aluminum oxide content is in the range of 6% to 12%.

The calculation formula of the silica-alumina ratio is as follows.

$$Silica \text{-} alumina ratio = \frac{\frac{a ( {SiO}_{2} content \% )}{60.09 ( Molecular weight of {SiO}_{2} )}}{\frac{b ( {Al}_{2} {O}_{3} content \% )}{101.96 ( Molecular weight of {Al}_{2} {O}_{3} )}} > 10 \qquad [ Expression 1 ]$$

For the composition for preventing and treating mastitis for livestock animals according to the present invention, in short, a rock powder in which silicic acid content is 60% or more and aluminum oxide content is in the range of 6% to 12% is effectively preferred. Moreover, the rock powder in which silicic acid content is 60% or more and aluminum oxide content is in the range of 6% to 12% with the silica-alumina ratio being more than 10 is more effectively preferred. Such a rock powder can be obtained by smashing one kind of rock or by preparing multiple kinds of rock powders. Furthermore, such a rock powder can be in the form of granules or pellets.

The required amount of the composition for preventing and treating mastitis for livestock animals according to the present invention is mixed with feed of the dairy cattle or livestock animals for being milked and given to them. Moreover, if the amount of this composition for preventing and treating mastitis given to the dairy cattle or livestock animals falls below 50 g per day for one cow, the effect cannot be expected for the amount falling below 50 g. Preferably, such an amount is 50 g or more per day.

The composition for preventing and treating mastitis for livestock animals according to the present invention was actually applied to the dairy cattle in a stock farm, and success or failure of its effect (result) was examined. The result of the examination is as shown in the following Table 1. Samplings of the milk were taken on Dec. 8 and 22, 2010, and after the samplings on December 22, silage with high concentration of nitrate nitrogen was given to the dairy cattle. Then, after samplings of the milk on Jan. 7, 2011, about 50 g of the composition for preventing and treating mastitis for livestock animals according to the present invention was given to the dairy cattle a day, and samplings of the milk were taken on February 10. In the concerned composition given in this examination, the silicic acid content was 60% or more and the aluminum oxide content was in the range of 6% to 12%, and the silica-alumina ratio in the concerned composition was more than 10. The stock farm which cooperated for this examination was Komatsu Stock Farm (Shikama-cho, Kami-gun, Miyagi), Shikama Kikaika Dairy and Farm Cooperative. The date of the examination: about 2 months from Dec. 8, 2010 to Feb. 10, 2011. Dairy Cooperative, CS Factory Name: ZEN-NOH Miyagi, Michinoku Milk Co., Ltd.

TABLE 1

| | Fat | Protein | Lactose | Solids-not-fat | Urea | Somatic | Number |
|---|---|---|---|---|---|---|---|
| Date of the | content | content | content | content | nitrogen | cell count | of bacteria |
| examination | (%) | (%) | (%) | (%) | (mg/dl) | (thousand/ml) | (thousand/ml) |
| Dec. 8, 2010 | 4.01 | 3.38 | 4.41 | 8.79 | 9.4 | 111 | 5 |
| Dec. 22, 2010 | 4.04 | 3.37 | 4.39 | 8.77 | 11.8 | 147 | 6 |
| Jan. 7, 2011 | 4.09 | 3.33 | 4.39 | 8.73 | 11.2 | 226 | 9 |
| Feb. 10, 2011 | 4.10 | 3.39 | 4.46 | 8.85 | 8.4 | 138 | 9 |
| Rate of change (%) | 100.2 | 101.8 | 101.6 | 101.4 | 70.0 | 61.1 | 100.0 |

As obvious from the above described Table 1, urea nitrogen and the somatic cell count decreased sharply by mixing the composition for preventing and treating mastitis for livestock animals according to the present invention with the feed and giving the mixture to the dairy cattle, which results in a direction of improving the factor for causing mastitis considerably, and it was confirmed that the composition of the present invention exhibits an effect for the prevention and treatment of mastitis and is useful.

That is, silicic acid and aluminum oxide contained in the composition of the present invention chemically adsorb nitrogen compounds including ammonia nitrogen and nitrate nitrogen generated in rumen, whereby the over-absorption of the nitrogen compounds through the digestive tract can be suppressed. At the same time, minor elements contained in the silicic acid of the underground mining act to fully elicit the activity of a nutritional component capable of suppressing mastitis, e.g. vitamin A, vitamin E, copper, zinc and manganese obtained from the feed. In this manner, mastitis can be suppressed in a nutritional manner. Silicic acid helps the minor elements being utilized effectively by holding them chemically in the digestive tract. At the same time, silicic acid adsorbs nitrate nitrogen to prevent its outflow and suppresses a bad influence of aluminum oxide to work for taking a turn for the better.

Moreover, the composition of the present invention increases the content of minerals in the excrement by being mixed with the feed and given to the animals or ingested by the animals, which accelerates the speed of fermentation of the excrement so that good quality of manure can be produced. Also, it has become difficult for nitrate nitrogen to flow from the excrement, which can reduce contamination of grass or groundwater by nitrate nitrogen.

Furthermore, other kind of minerals will be ingested by the livestock animals such as the milked cows by mixing the composition of the present invention with the feed and providing the animals with the mixture, thereby improving fecundity of the livestock animals as well as keeping other diseases away.

Next, success/failure for the effect (result) was examined, which was obtained by changing the amount of the composition for preventing and treating mastitis for livestock animals according to the present invention given to the animals. The result of the examination is as shown in the following Table 2. The following Table 2 is a graph plotting the average of the somatic cell count contained in the samplings of milk taken daily from Apr. 1 to Jul. 18, 2011 targeted for 200 dairy cattle in a stock farm in Chiba. In a period of time from May 14 to May 29, 2011, about 35 g of the composition for preventing and treating mastitis for livestock animals according to the present invention was given to the animals a day, in a period of time from May 30 to June 16, the concerned composition was not given to the animals, and in a period of time from June 17 to July 18, about 50 g of the concerned composition was given to the animals a day. In the concerned composition given in this examination, silicic acid content was 60% or more and aluminum oxide content was in the range of 6% to 12%, and the silica-alumina ratio in the concerned composition was more than 10. Moreover, a farming mode of the subject dairy cattle was free barn, and a feeding mode was TMR (Total Mixed Rations).

As obvious from the above described Table 2, decrement of the somatic cell count increased depending on the gain in the amount of the composition for preventing and treating mastitis for livestock animals according to the present invention given to the animals.

In particular, when the amount of the composition given to the animals was 50 g, the somatic cell count decreased sharply. In this examination, depending on the gain in the amount of the concerned composition given to the animals, decrement of the urea nitrogen in the samplings of the milk also increased, and the result was obtained in which urea nitrogen decreased sharply when the amount of the composition given to the animals was 50 g. Thus, it was confirmed that the composition for preventing and treating mastitis for livestock animals according to the present invention contributes a great deal to the prevention and treatment of mastitis.

Next, with regard to the effect (result) obtained when the composition for preventing and treating mastitis for livestock animals according to the present invention was given to the animals, the number of dairy cattle with mastitis, the number of dairy cattle shipped, and the volume of bulk milk in a stock farm in Tochigi were examined. The following Table 3 is a graph plotting the number of dairy cattle with mastitis in the prescribed period of time, the following Table 4 is a graph plotting the number of dairy cattle shipped in the prescribed period of time, and the following Table 5 is a graph plotting the volume of bulk milk in the prescribed period of time.

The periods of time shown in the following Table 3 to Table 5 include a period of time from Mar. 1 to Apr. 30, 2011 and a period of time from Mar. 1 to Apr. 30, 2012. In 2011, about 700 dairy cattle were subjected to this examination, and the composition for preventing and treating mastitis for livestock animals according to the present invention was not given to them. In 2012, about 720 dairy cattle were subjected to this examination, and about 100 g of the concerned composition was given to them a day from April 1 to April 30. Also, in the concerned composition given in this examination, silicic acid content was 60% or more and aluminum oxide content was in the range of 6% to 12%, and the silica-alumina ratio in the concerned composition was more than 10. Moreover, a farming mode of the subject dairy cattle was free stall, and a feeding mode was TMR (Total Mixed Rations).

As obvious from the above described Table 3 to Table 5, the result was obtained in which the number of dairy cattle with mastitis decreased sharply by giving the animals the composition for preventing and treating mastitis for livestock animals according to the present invention, and the number of dairy cattle shipped and the volume of bulk milk increased sharply by giving the animals the concerned composition. Also, in this examination, the result was obtained in which the amount of urea nitrogen and the somatic cell count in the samplings of the milk decreased sharply by giving the animals the concerned composition. Thus, it was confirmed that the composition for preventing and treating mastitis for livestock animals according to the present invention contributes a great deal to the prevention and treatment of mastitis.

Also the result in which the composition for preventing and treating mastitis for livestock animals according to the present invention contributes to the prevention and treatment of mastitis in the livestock animals by being mixed with the feed and given to the animals was obtained in other examinations with conditions which are different from those in the above described Table 1 to Table 5. For example, the result was obtained in which when the composition with silicic acid content being less than 60%, aluminum oxide content being less than 6%, and silica-alumina ratio being less than 10 was mixed with the feed and given to the animals, the amount of urea nitrogen and the somatic cell count did not change much or increased conversely, and its effect was inferior to the effect obtained when the composition for preventing and treating mastitis for livestock animals according to the present invention was given to the animals.

Now, it is preferred that the rock which is the composition for preventing and treating mastitis for livestock animals according to the present invention have a three-layered structure of a silicic acid layer-an alumina layer-a silicic acid layer. Such a rock having a three-layered structure can include a smectite rock represented by montmorillonite, which is because the ability of adsorbing nitrogen compounds chemically may not be exercised fully even if silicic acid content is 60% or more, aluminum oxide content is in the range of 6% to 12%, and the silica-alumina ratio is more than 10 when the rock is a rock having a three-layered structure of a silicic acid layer-an alumina layer-a silicic acid layer. That is, when the rock which is the composition for preventing and treating mastitis for livestock animals according to the present invention has a three-layered structure of a silicic acid layer-an alumina layer-a silicic acid layer, the greater effect can be exhibited in the prevention and treatment of mastitis as compared to a rock without the concerned three-layered structure. This is reported, for example, in “Tsuchi no wakagaeri wo hakaru nendo noho, San Ra Tail no iryoku, by Hoji Kobayashi, Rural Culture Association, published on Sep. 30, 1990” (Clay Farming for Attempting Rejuvenation of Soil, Power of San Ra Tail, by Hoji Kobayashi, Rural Culture Association, published on Sep. 30, 1990).

Moreover, it is preferred that the rock which is the composition for preventing and treating mastitis for livestock animals according to the present invention be shale (siliceous shale) formed by including mainly phytoplankton, terrestrial plants, and carcasses of other organisms since such shale has a high tendency to have silicic acid content which is 60% or more, aluminum oxide content which is in the range of 6% to 12%, and the silica-alumina ratio which is more than 10 even without preparing multiple kinds of rock powders. Moreover, it is because, in addition to silicic acid and aluminum oxide, multiple types of minor elements are contained in a state of easily being eluted, which can increase the content of minerals in rumen or excrement.

Also, in terms of keeping silica-alumina ratio of the shale, it is preferred that the rock be shale obtained by underground mining as compared to strip mining.

### INDUSTRIAL APPLICABILITY

Although the composition for preventing and treating mastitis for livestock animals according to the present invention was examined (tested) with dairy cattle, it is natural that the concerned composition can be widely used generally as long as they are livestock animals to be milked.

1. A composition for preventing and treating mastitis for livestock animals, comprising a rock powder which contains silicic acid and aluminum oxide as active ingredients.

2. The composition for preventing and treating mastitis for livestock animals according to claim 1, wherein the silicic acid content is 60% or more and the aluminum oxide content is in the range of 6 to 12%.

3. The composition for preventing and treating mastitis for livestock animals according to claim 2, wherein the ratio of a value obtained by dividing the silicic acid content by the molecular weight of the silicic acid to a value obtained by dividing the aluminum oxide content by the molecular weight of the aluminum oxide is more than 10.

4. The composition for preventing and treating mastitis for livestock animals according to claim 3, wherein the rock powder has a three-layered structure of a silicic acid layer-an alumina layer-a silicic acid layer.

5. The composition for preventing and treating mastitis for livestock animals according to claim 4, wherein the rock is shale which mainly includes sediment from organisms.

6. The composition for preventing and treating mastitis for livestock animals according to claim 1, wherein the powder of the active ingredients is in the form of granules or pellets.
